SEQUENCE LISTING (8 sequences)

SEQ ID NO: 1 - DNA, 64 bp, Artificial
  NF-kB-TAR sequence
gggtctctctggttagcatcctgaaactgttttaaggttggccgatgtagctagggaacccact

SEQ ID NO: 2 - DNA, 19 bp, Artificial
  siRNA2 encoding sequence
tattagagcaacctaaaca

SEQ ID NO: 3 - DNA, 24 bp, artificial
  DNA probe containing binding site for NF-kB upstream primer
gccatggggggatccccgaagtcc

SEQ ID NO: 4 - DNA, 24 bp, artificial
  DNA probe containing binding site for NF-kB reverse primer
ggacttcggggatccccccatggc

SEQ ID NO: 5 - RNA, 65 nt, artificial
  U6/TAR-a-p50 vector HIV1 TAR with NF-kB-TAR sequence
gggucucucugguuagcauccugaaacuguuuuaagguuggccgaugcuagcuagggaacccacu

SEQ ID NO: 6 - RNA, 198 nt, artificial
  pAV7SL-a-p50
gccgggcgcgguggcgcgugccuguagucccagcuacucgggaggcugaggcuggaggaucgcuugaguccaggaguucugggcuguagugcgcuauauccugaaacuguuuuaagguuggccgauauagccacugcacuccagccugggcaacauagcgagaccccgucucgcuagagcggacuucgguccgcuuuu

SEQ ID NO: 7 - RNA, 55 nt, artificial
  HIV 1-TAR transcript
ucugguuagaccagaucugagccugggagcucucuggcuagcuagggaacccacu

SEQ ID NO: 8 - DNA, 10 bp, artificial
  Immunostimulatory motif
aacgttcgag

(19) United States
(12) Patent Application Publication
(10) Pub. No.: US 2005/0250106 A1
(43) Pub. Date: Nov. 10, 2005

(54) Gene knock-down by intracellular expression of aptamers

(76) Inventors: David Epstein, Belmont, MA (US); Harold Nicholas Marsh, Charlestown, MA (US); Shannon Pendergrast, Cambridge, MA (US); Kristin Thompson, Arlington, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/831,632

(22) Filed: Apr. 23, 2004

Related U.S. Application Data
(60) Provisional application No. 60/465,853, filed on Apr. 24, 2003.

(51) Int. Cl. C12Q 1/68; C07H 21/02; A61K 48/00

(52) U.S. Cl. 435/6; 514/44; 536/23.1

(57) ABSTRACT

Materials and methods are provided for target validation by gene knock-down with intracellularly expressed aptamers and siRNAs. The aptamers produced by the materials and methods of the invention are useful in target validation for therapeutics development.

### REFERENCE TO RELATED APPLICATIONS

The present non-provisional patent application claims priority under 35 U.S.C. §119(e) to U.S. provisional patent application Ser. No. 60/465, 853 filed Apr. 24, 2003, and is related to U.S. provisional patent application Ser. No. 60/442,249 filed Jan. 23, 2002 (now abandoned), each of which is hereby incorporated by reference in its entirety.

### FIELD OF THE INVENTION

The present invention relates generally to the field of nucleic acids and more particularly to materials and methods of gene regulation by the intracellular expression of aptamers having specificity to a protein target. The invention further relates to target validation materials and methods for the simultaneous expression of aptamers and small interfering RNAs (siRNAs) to effect maximal knock-down of gene expression with resulting knock-down of protein activity.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial (therapeutic) leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 kDa; antibody: 150 kDa), a weekly dose of aptamer may be delivered by injection in a volume of less than 0.5 ml. Aptamer bioavailability via subcutaneous administration is >80% in monkey studies (Tucker et al., J. Chromatography B. 732: 203-212, 1999). In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Genome-wide sequencing projects have led to the uncovering of thousands of new genes of unknown function. Despite the flood of new gene sequence information, the role of well-studied genes in complex multi-gene dependent processes, such as disease pathology, remains elusive. The most effective methods used for facilitating gene function elucidation are those that suppress gene activity. Among these, the specific-down regulation of gene expression in cells is a powerful method for elucidating a gene's function. The most commonly used method for suppressing gene expression is the elimination of messenger RNA by RNA interference (RNAi) or antisense. RNAi has become a widely used tool for the suppression of gene activity in both invertebrates and plants, and with the advent of small interfering RNA (siRNA) techniques, in mammalian cells. The siRNA molecules bind to a protein complex, called the RNA-induced silencing complex. This complex contains a helicase activity that unwinds the two strands of siRNA molecules allowing the antisense strand of the siRNA to bind to the targeted mRNA molecule and an endonuclease activity that hydrolyzes the target mRNA at the site where the antisense strand is bound (Schwarz et al. (2002)Mol. Cell. 10, 537-48).

Although a powerful method, there are limits to siRNA techniques. Firstly, siRNAs don't always promote complete degradation of mRNA as only a subset of sites on an mRNA are good target sites for siRNA molecules, probably because of RNA secondary structure. If even a small amount of mRNA survives it may be able to produce sufficient amounts of protein for significant activity. Secondly, recent studies indicate that siRNAs can have adverse effects by activating sensors in the interferon response pathway or other non-specific genes. Finally, siRNA (as well as anti-sense or gene knock-out strategies) may completely or severely deplete targeted protein levels. Since many proteins exist in multi-protein complexes that may be involved in multiple functional pathways, deleting the protein will likely have pleiotropic effects that are not specific to the relevant pathway.

There is a need for methods for the elucidation of gene function and target validation, particularly in relation to disease progression. Accordingly, it would be beneficial to have materials and methods to down-regulate gene expression to assist in determining a gene's function and validate targets for therapeutics. The present invention provides materials and methods to meet these and other needs.

### BRIEF DESCRIPTION OF THE DRAWINGS

FIG. 1 is a schematic representation of the in vitro aptamer selection (SELEX™) process from pools of random sequence oligonucleotides.

FIG. 2A is a schematic of an RNA transcript (SEQ ID NO:6) generated from 7SL-NF-κB in which the 7SL sequence is shown in bold text, the aptamer sequence shown underlined, and the terminator sequence shown in italics; FIG. 2B (Panel 1, left side) is a schematic of the HIV-1 TAR transcript (SEQ ID NO:7) consisting of a 59 nucleotide stable stem and loop structure; the DNA encoding the stem also encodes the bipartite IST element showing the IST sequence is presented in normal text and the sequence most important for Tat binding is shown in italics; FIG. 2B (Panel 2, right side) is schematic of the HIV-1 TAR with NF-κB-TAR sequence (SEQ ID NO:5), in which the Tat binding site is replaced with the similarly shaped p50 aptamer which sequence is shown underlined.

FIG. 3A is a bar graph of fluorescence (RLU) produced by 7SL NF-κB transfectants treated with TNF alpha at 20 ng/mL (+) or medium alone (−) showing reduced NF-κB-luciferase activity compared to vehicle controls (293 cells were co-transfected with NF-κB-luciferase reporter and 7sl or 7SL-NF-κB); FIG. 3B shows a bar graph of fluorescence (RLU) produced by U6 TAR or U6 TAR NF-κB transfectants treated with TNF alpha at 20 ng/mL (+) or medium alone (−).

FIG. 4A is a bar graph of normalized results of a Western blot of cells transfected with control p-Silencer-2.0 plasmid (CONTROL) or p-Silencer-2.0-U6-siRNA2 SEQ2 (SEQ ID No. 3); FIG. 4B is a bar graph of results of a NF-κB-dependent luciferase assay of cells transfected with NF-κB-dependent reporter plasmid and p-Silencer-2.0 (siRNA CON) or p-Silencer-2.0-U6-siRNA2 (siRNA2).

FIG. 5 is a bar graph of results of luciferase assay showing that NF-κB activity is most significantly inhibited in the presence of both p50-specific siRNA and p50-specific aptamer.

FIG. 6 is a schematic of a DNA vector having an expression cassette region comprising a promoter, IST, aptamer, siRNA or aptamer/siRNA insert, IST, and terminator regions, and the corresponding RNA transcript.

FIG. 7 is a schematic of one embodiment of a DNA vector having two tandem expression cassette regions comprising a promoter, IST, aptamer or siRNA insert, IST, aptamer or siRNA insert, IST, and terminator regions, and the corresponding RNA transcript.

### SUMMARY OF THE INVENTION

The present invention provides materials and methods to down-regulate gene expression in cells to assist in determining gene function as a tool for target validation for the development of novel therapeutics.

In one embodiment, the present invention provides materials and methods for simultaneous expression of intracellular aptamers and concurrent expression of small interfering RNAs (siRNAs) to maximally knock-down gene activity to assist in determining gene function and as a tool for target validation for the development of novel therapeutics.

In one embodiment, the present invention provides expression vectors for the intracellular expression of aptamers specific to target proteins and expression vectors for the intracellular expression of siRNAs.

In one embodiment, the present invention provides expression vectors for the intracellular expression of aptamers specific to nuclear factor κB (NF-κB) transcription factor which is central to the overall immune response where it mediates both the activation and survival of T cells.

In one embodiment, the present invention provides expression vectors for the intracellular expression of nucleic acids to target proteins, said nucleic acids can be aptamers or siRNAs, or both. In one embodiment, the present invention provides expression vectors that have a transcription promoter, an inducer of short transcripts (IST) region from HIV-1 Trans-activation region (TAR), and a transcription termination region.

In one embodiment, the present invention provides methods to increase the over-expression of intracellular nucleic acids that act either in the cell nucleus or cytoplasm, said nucleic acids including aptamers or siRNAs or both. The method includes expressing the nucleic acid to bind to intracellular proteins in a vector including a transcription promoter, an IST sequence from HIV-1 TAR, a transcription termination region, and at least one nucleic acid aptamer or siRNA, or a combination of both, arranged in tandem along the vector sequence.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

The SELEX™ Method

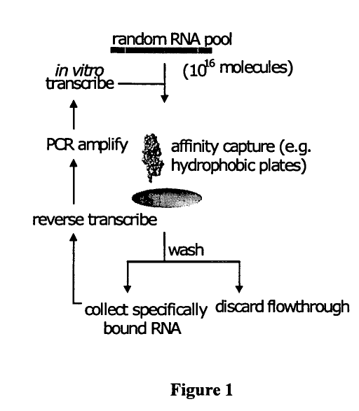

A suitable method for generating an aptamer for use in the materials and methods of the present invention is with the process entitled “Systematic Evolution of Ligands by Exponential Enrichment” (“SELEX™”) generally depicted in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores, sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs. See, e.g., U.S. Pat. Nos. 5,958,691; 5,660,985; 5,958,691; 5,698,687; 5,817,635; and 5,672,695, PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 μmole) scale synthesis will yield 10¹⁵-10¹⁶ individual template molecules, sufficient for most SELEX experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain sub portions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, see U.S. Pat. No. 5,580,737 which discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

The identification of nucleic acid ligands to small, flexible peptides via the SELEX method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase or modified T7 RNA polymerases and purified. In one example, the 5′-fixed:random:3′-fixed sequence is separated by random sequence having 30 to 50 nucleotides.

The aptamers with specificity and binding affinity to the targets of the present invention are selected by the SELEX process described above. As part of the SELEX process the sequences selected to bind to the target are then optionally minimized to determine the minimal sequence having binding affinity, and optimized by performing random or directed mutagenesis of the minimized sequence to determine if increases of affinity or alternatively which positions in the sequence are essential for binding activity. Additionally, selections can be performed with sequences incorporating modified sequences to stabilize the aptamer molecules against degradation in vivo.

Intracellular Expression of Aptamers and siRNAs for Gene Knock-Down

The present invention provides methods to effect gene-knock down as a tool for target validation in therapeutics development. The present invention further provides aptamers to inhibit intracellular target protein activity in mammalian cells. The present invention also provides vectors for the over-expression of intracellular nucleic acids that bind to intracellular targets, said targets can be proteins in the nucleus or in the cytoplasm, as well as extra-cellular targets. The materials and methods of the present invention are useful to determine the activity of genes and are thus useful alone or in combination with known methods as target validation tools in the development of therapeutics.

The methods of the present invention are used to produce nucleic-acid-derived aptamers to regulate intracellular protein activity. Aptamers specific to either RNA binding proteins (decoy aptamers) or non-RNA binding proteins (non-decoy aptamers), are generated, e.g., by the SELEX™ process described above. These aptamers selected to bind with high specificity and affinity and have the ability to knock-out intracellular protein activity. For instance, aptamers that recognize the cytoplasmic domain of the B2 integrin, leukocyte function-associated molecule-1 (LFA-1) have been isolated (Famulok et al., (2001) Chem. Biol. 8, 931-9). LFA-1 mediates the adhesion of leukocytes in immune responses by binding to intracellular adhesion molecules. Intracellular expression of anti-LFA-1 aptamers inhibits LFA-1 activity, as measured by a decrease in cell-adhesion. Additionally, an anti-cytohesin 1 aptamer has been used to block the intracellular function of cytohesin-1, a guanine-nucleotide-exchange factor that is thought to regulate the adhesion of LFA-1 to ICAM-1 (Mayer et al. (2001), Proc. Natl. Acad. Sci. USA 98, 4961-5). Moreover, aptamer-based cytohesin-1 inhibition results in a decrease in cell adhesion and cytoskeletal rearrangement. The present invention provides aptamers to inhibit intracellular protein activity. The present invention also provides methods that combine the use of intracellularly expressed aptamers and siRNAs to further increase gene knock-down. These methods are useful as target validation tools in the development of therapeutics.

Intracellular Aptamer and siRNA Expression Vectors

The present invention provides materials and methods to down-regulate gene expression in cells to assist in determining gene function as a tool for target validation for the development of novel therapeutics. The present invention provides expression vectors for the intracellular expression of aptamers specific to nuclear factor κB (NF-κB) transcription factor, which is central to the overall immune response where it mediates both the activation and survival of T cells. The expression vectors of the present invention comprise a transcription promoter, an inducer of short transcripts (IST) region from HIV-1 Trans-activation region (TAR), and a transcription termination region. The nuclear directing intracellular expression vectors of the present invention can be used for the intracellular over-expression of nucleic acids to target proteins, wherein the nucleic acids can be aptamers or siRNAs, or combinations of both.

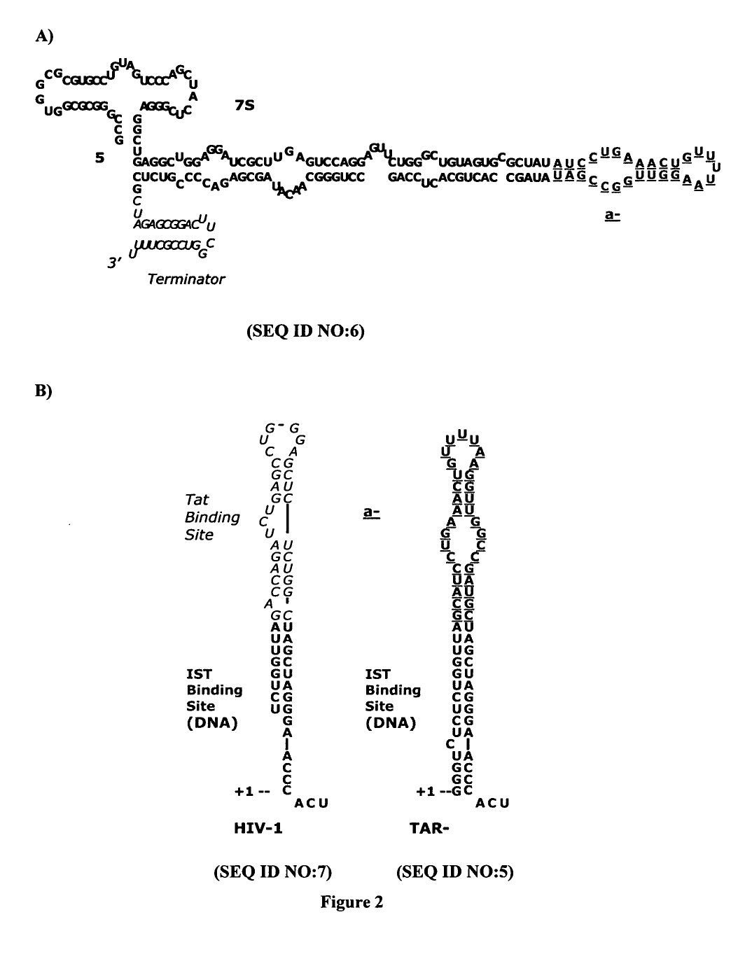

The intracellular over-expression vectors of the invention include pAV7SL and U6/TAR-derived vectors. pAV7SL uses sequences derived from the natural 7SL siRNA to stabilize the transcript and direct it to the cytoplasm. U6/TAR is novel and based on the construct U6-HIV/LTR that expresses the natural, predominantly nuclear HIV-1 TAR aptamer from the U6 promoter. The top part of the TAR contains a natural aptamer that binds the HIV-1 Tat protein. This natural aptamer can be replaced with a different RNA sequence such as an aptamer or siRNA molecule (FIG. 2B). This RNA sequence is then expressed to high levels within the cell. This construct offers several advantages over other published nuclear-directing constructs. Firstly, the RNA vehicle generated is simple compared to tRNA-based constructs (Good et al. (1997) Gene Ther. 4, 45-54, Bertrand et al. (1997) RNA 3, 75-88.) Secondly, the TAR has been reported to be very stable; with a half-life of from 2-3 hours in vivo (Pfeifer et al. (1991) J. Biol. Chem. 266, 14620-14626.). Finally, the DNA encoding the bottom third of the TAR contains an IST (Inducer of Short Transcripts) element. The IST element has been shown to dramatically increase the amount of constitutively produced short transcripts from a variety of promoters including the U6 promoter (Ratnasbapathy et al. (1990) Genes Dev. 4, 2061-2074). Thus, this plasmid produces significantly more aptamer than other vectors containing snRNA promoters (U6 or U1). 7SL-a-p50 is more effective than TAR-a-p50 at inhibiting NF-κB activity (29% vs. 51%). This result is believed to be due to differences in stability/folding of the RNA transcripts or to differences in accessibility of the target in the cytoplasm versus the nucleus.

The present invention provides vectors for the intracellular over-expression of an anti-NF-κB aptamer, a-p50 (Lebruska and Maher (1999), Biochemistry 38, 3168-3174). Plasmid U6/TAR-a-p50 contains a U6 promoter followed by the TAR-a-p50 sequence. The plasmid vector U6/TAR-a-p50 of the present invention was made by inserting the nuclear factor κB-trans-activation region (NF-κB-TAR) sequence, GGGTCTCTCTGGTTAGCATCCTGAAACTGTTTTAAGGTTGGCCGATGTAGCTAGGGAACCCACT (SEQ ID NO: 1) (flanked by XhoI/BamHI sites and generated by PCR), into the XhoI/BamHI restriction sites of the plasmid MYHIV (. The HIV-1 promoter sequence was then replaced by the pol III U6 promoter by inserting a PCR-generated fragment into EcoRI/XhoI linearized plasmid.

Plasmid pAV7SL-a-p50 of the present invention was made by inserting a PCR-generated fragment consisting of a-p50 (see FIG. 2) into the SalI-XbaI sites of pAV7SL. pSilencer-2.0-U6-siRNA2 was made by inserting the fragment encoding siRNA2: TATTAGAGCAACCTAAACA (SEQ ID NO:2) into the XbaI/BamHI sites of the vector pSilencer (Ambion) vector.

The present invention further provides methods to increase intracellular nucleic acid expression using the intracellular over-expression vectors of the present invention that include an IST sequence to produce significant increases in intracellular nucleic acid aptamers and/or siRNAs to effect gene knock-down. The present invention provides intracellular nucleic acid expression vectors that allow for the simultaneous expression of aptamers and siRNAs from the same intracellular expression vector to achieve a higher level of gene-knock down than with either tool alone. There are theoretical and practical reasons for combining siRNA and aptamers. They are both RNA molecules and can be introduced in similar ways (i.e. expressed from plasmids or in vitro transcribed and directly transfected). They work by inhibiting different levels of the protein expression pathway, and thus, they are unlikely to interfere with each other and more likely to synergize.

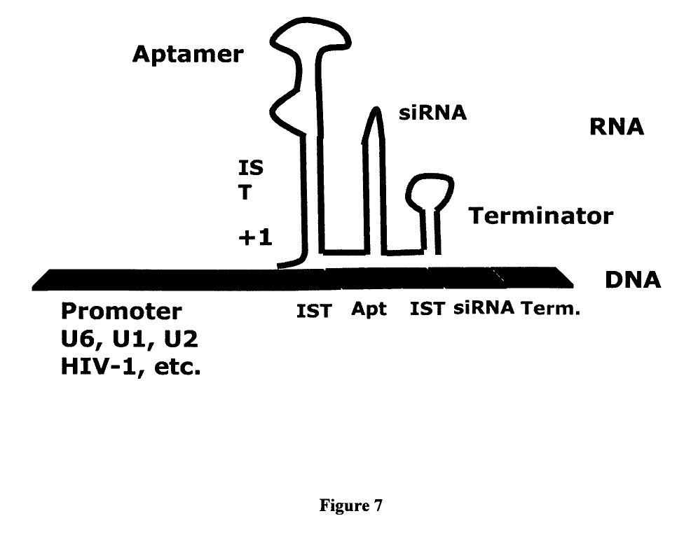

The present invention provides materials and methods for simultaneous expression of intracellular aptamers and concurrent expression of small interfering RNAs (siRNAs) to maximally knock-down gene activity to assist in determining gene function and as a tool for target validation for the development of novel therapeutics (FIG. 7).

The present invention provides methods to increase the over-expression of intracellular nucleic acids that act either in the cell nucleus or cytoplasm, said nucleic acids including aptamers and siRNAs. The method includes expressing the nucleic acid to bind to intracellular proteins in a vector including a transcription promoter, an IST sequence from HIV-1 TAR, a transcription termination region, and at least one nucleic acid aptamer or siRNA, or a combination of both, arranged in tandem along the vector sequence.

The methods of the present invention demonstrate that the simultaneous expression of siRNA and RNA aptamers in cells inhibits a protein's expression better than either method alone. This technique can be useful in situations where a complete knock-out is desired and neither technique is capable of effecting the desired level of gene knock-down alone.

Aptamers Having Immunostimulatory Motifs

Recognition of bacterial DNA by the vertebrate immune system is based on the recognition of unmethylated CG dinucleotides in particular sequence contexts (“CpG motifs”). One receptor that recognizes such a motif is Toll-like receptor 9 (“TLR 9”), a member of a family of Toll-like receptors (˜10 members) that participate in the innate immune response by recognizing distinct microbial components. TLR 9 binds unmethylated oligodeoxynucleotide (ODN) CpG sequences in a sequence-specific manner. The recognition of CpG motifs triggers defense mechanisms leading to innate and ultimately acquired immune responses. For example, activation of TLR 9 in mice induces activation of antigen presenting cells, up regulation of MHC class I and II molecules and expression of important costimulatory molecules and cytokines including IL-12 and IL-23. This activation both directly and indirectly enhances B and T cell responses, including robust up regulation of the TH1 cytokine IFN-gamma. Collectively, the response to CpG sequences leads to: protection against infectious diseases, improved immune response to vaccines, an effective response against asthma, and improved antibody-dependent cell-mediated cytotoxicity. Thus, CpG ODN's can provide protection against infectious diseases, function as immuno-adjuvants or cancer therapeutics (monotherapy or in combination with mAb or other therapies), and can decrease asthma and allergic response.

A variety of different classes of CpG motifs have been identified, each resulting upon recognition in a different cascade of events, release of cytokines and other molecules, and activation of certain cell types. See, e.g., CpG Motifs in Bacterial DNA and Their Immune Effects, Annu. Rev. Immunol. 2002, 20:709-760, incorporated herein by reference. Additional immunostimulatory motifs are disclosed in the following U.S. patents, each of which is incorporated herein by reference: U.S. Pat. Nos. 6,207,646; 6,239,116; 6,429,199; 6,214,806; 6,653,292; 6,426,434; 6,514,948 and 6,498,148. Any of these CpG or other immunostimulatory motif can be incorporated into the sequence of an aptamer to be used in the nucleic acids of the present invention, the choice dependent on the disease or disorder to be treated. Preferred immunostimulatory motifs are as follows (shown the 5′ to 3′ left to right) wherein “r” designates a purine, “y” designates a pyrimidine, and “X” designates any nucleotide: AACGTTCGAG (SEQ ID NO:8); AACGTT; ACGT, rCGy; rrCGyy, XCGX, XXCGXX, and X₁X₂CGY₁Y₂ wherein X₁ is G or A, X₂ is not C, Y₁ is not G and Y₂ is preferably T.

In those instances where a CpG motif is incorporated into an aptamer that binds to a specific target other than a target known to bind to CpG motifs and upon binding stimulate an immune response (a “non-CpG target”), the CpG is preferably located in a non-essential region of the aptamer. Non-essential regions of aptamers can be identified by site-directed mutagenesis, deletion analyses and/or substitution analyses. However, any location that does not significantly interfere with the ability of the aptamer to bind to the non-CpG target may be used. In addition to being embedded within the aptamer sequence, the CpG motif may be appended to either or both of the 5′ and 3′ ends or otherwise attached to the aptamer. Any location or means of attachment may be used so long as the ability of the aptamer to bind to the non-CpG target is not significantly interfered with.

As used herein, “stimulation of an immune response” can mean either (1) the induction of a specific response (e.g., induction of a Th1 response) or of the production of certain molecules or (2) the inhibition or suppression of a specific response (e.g., inhibition or suppression of the Th2 response) or of certain molecules.

CpG motifs can be incorporated or appended to an aptamer against any target including but not limited to: PDGF, IgE, IgE Fcε R1, TNFa, PSMA, CTLA4, PD-1, PD-L1, PD-L2, FcRIIB, BTLA, TIM-3, CD11b, CD-11c, BAFF, B7-X, CD19, CD20, CD25 AND CD33.

By incorporating CpG motifs into aptamers specifically targeting solid tumors these aptamers can be used to activate the immune system through the recruitment of antigen presenting cells that have taken up tumor derived material, enhance their maturation and migration to local lymph nodes and increase priming of tumor specific T-cells. This is especially relevant where aptamers deliver cytotoxic payload and result in cell death (such as a PSMA aptamer containing a CpG motif). Such CpG motif containing aptamers can also induce tumor-specific memory response (prophylactic use). In addition, the IFP lowering and pericyte recruitment blocking effects of a PDGF-B aptamer combined with the increased immune response observed upon CpG administration represents a potent therapeutic for cancer. Thus, aptamers with incorporated, appended or embedded CpG motifs represent a novel class of anti-cancer compounds such that when administered they can lead to a significant de-bulking of the tumor through two mechanisms: first, through activation of tumor specific T-cells within the tumor bed and second, through the intended mechanism-based action of the aptamer pharmacophore.

Pharmaceutical Compositions

The invention also includes pharmaceutical compositions and the uses of pharmaceutical compositions containing oligonucleotides comprising aptamer and siRNA molecules to affect gene knock-down in a patient. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers specifically bind.

For example, the target is a protein involved with a pathology, for example, the target protein causes the pathology.

Compositions of the invention can be used in a method for treating a patient or subject having a pathology. The method involves administering to the patient or subject a composition comprising aptamers that bind a target (e.g., a protein) involved with the pathology, so that binding of the composition to the target alters the biological function of the target, thereby treating the pathology.

The patient or subject having a pathology, e.g. the patient or subject treated by the methods of this invention can be a mammal, or more particularly, a human.

In practice, the compounds or their pharmaceutically acceptable salts, are administered in amounts which will be sufficient to exert their desired biological activity, e.g., inhibiting the binding of a cytokine to its receptor.

The siRNA-aptamer composition of the invention may contain, for example, more than one siRNA-aptamer. In some examples, an siRNA-aptamer composition of the invention, containing one or more compounds of the invention, is administered in combination with another useful composition such as an anti-inflammatory agent, an immunosuppressant, an antiviral agent, or the like. Furthermore, the compounds of the invention may be administered in combination with a chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

“Combination therapy” (or “co-therapy”) includes the administration of an siRNA-aptamer composition of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical. “Combination therapy” also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The compounds of the invention and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, cremes, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the siRNA-aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 5000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### NF-κB Gene Knock-Down with Aptamers and siRNA

The methods of the present invention, which utilize an aptamer that has been shown previously to inhibit the DNA binding activity of the p50 subunit of the NF-κB transcription factor in yeast (Lebruska, et al., (1999) Biochemistry, 38, 3168-3174), were used for the first time to knock-down NFκB activity in mammalian cells. Furthermore, NF-κB activity was knocked-down with siRNA. Both aptamers and siRNAs were found to have knock-down activity in a similar manner, and when used in combination the two methods work better than either method alone, leading to a 90% knock-down of activity.

Plasmids. U6/TAR-a-p50 contains a U6 promoter followed by the TAR-a-p50 sequence. U6/TAR-a-p50 was made by inserting the NF-κB-TAR sequence (SEQ ID No.1), GGGTCTCTCTGGTTAGCATCCTGAAACTGTTTTAAGGTTGGCCGATGTAGCTAGGGAACCCACT (flanked by XhoI/BamHI sites and generated by PCR), into the XhoI/BamHI restriction sites of the plasmid MYHIV. The HIV-1 promoter sequence was then replaced by the pol III U6 promoter by inserting a PCR-generated fragment into EcoRI/XhoI linearized plasmid.

pAV7SL-a-p50 was made by inserting a PCR-generated fragment consisting of a-p50 into the SalI-XbaI sites of pAV7SL. P-Silencer-2.0-U6-siRNA2 was made by inserting the fragment encoding siRNA2 (SEQ ID No. 2): TATTAGAGCAACCTAAACA into the XbaI/BamHI sites of the vector pSilencer (Ambion).

Design of short interfering RNA (siRNA). Short interfering RNA (siRNA) targeting NF-κB p50 was designed according to Tuschl's design rules as described in Elbashir et al., Nature 411: 494-498 (2001) and Harboth et al., Antisense Nucleic Acid Drug Dev. 13: 83-106 (2003), both of which are incorporated herein by reference. See also: http://www.rockefeller.edu/labheads/tuschl/sirna.html. The sequence and its complement was BLAST searched against the human genome to ensure that only NF-κB p50 was targeted. Six sequences were designed. The sequence that showed the greatest ability to reduce protein levels by Western blot after in vitro transcription and transfection (see below) was siRNA sequence 2 (SEQ ID No. 2): TATTAGAGCAACCTAAACA.

Western Blot analysis of Cells transfected with in vitro transcribed siRNAs. HeLa cells were cultivated in DMEM supplemented with 10% fetal bovine serum in 12 well culture dishes at a density of 100,000 cells/well 24 hours before transfection. siRNAs were in vitro transcribed using Ambion's (Austin, Tex.) Silencer siRNA Construction kit. siRNA was transfected into HeLa cells using siPORT Lipid (Ambion, Austin, Tex.) with a siRNA concentration of 40 nM and 50 nM. After a 48 hour incubation in a 37° C. incubator, the HeLa cells were stimulated with human TNF alpha at a concentration of 10 ng/ml for four hours. The cells were extracted over ice in extraction buffer (10 mM Tris pH 7.5, 100 mM NaCl, 0.125% NP-40, 0.875% Brij 97, 1.5 mM Na-Vanadate, 1 mini-EDTA free protease inhibitor tablet from Roche). Total protein concentration levels were determined using a BCA Protein Assay kit (Pierce). 2 μg of total protein was loaded into a 15 well Invitrogen NuPAGE Novex 10% Bis-Tris gel (Invitrogen, Carlsbad, Calif.) and run in 1×MES Buffer (20×MES: 1M MES, 1M Tris Base, 69.3 mM SDS, 20.5 mM EDTA at pH 7.3) for 35 minutes at a constant 200 volts. Transfer of 1 hour at a constant 30 volts onto nitrocellulose film using 1×NuPAGE Transfer Buffer (20×Transfer Buffer: 500 mM Bicine, 500 mM Bis-Tris, 20.5 mM EDTA, 1 mM Chlorobutanol at pH 7.2).

Western blots used antibodies to NF-κB-p50 (Upstate Cell Signaling Solutions, Waltham, Mass.) and TFIII-B at a dilution of 1:5000. The TFIII-B antibody serves as an internal control to normalize for variations in protein loading. The nitrocellulose film was incubated overnight in primary antibody followed by one hour secondary anti-rabbit antibody incubation. Imaging is done with ECL Western Blot kit (Amersham, Arlington Heights, Ill.) and the fluorescence was read at on the STORM machine according to Amersham protocol.

Western Blot analysis of cells transfected with siRNA expressing constructs. HEK293 cells were plated in 12 well plates at 100,000 cells per well in 1 ml of DMEM medium supplemented with 10% FBS and P/S. 450 ng of siRNA plasmid and 50 ng of GFP plasmid were transfected by Fugene 6. 48 hours later TNF-alpha (Calbiochem, San Diego, Calif.) was added at 10 ng/ml concentration for 6 hours. Cell extracts were generated by lysis in extraction buffer (10 mM Tris pH 7.5, 150 mM NaCl, 0.125% NP-40, 0.875% Brij 97, 1.5 mM Na Vanadate, and 2 mM EDTA protease free inhibitor). 7.5 μg protein (as determined by Bradford assay) of each sample was run on a NuPAGE 10% Bis Tris Gel using the Invitrogen Gel running system. Western Blot analysis was performed as above.

Electrophoretic Mobility (Gel) Shift Assay. A DNA probe containing the binding site for NF-κB was constructed using the following primers (SEQ ID No. 3): 5′-GCC ATG GGG GGA TCC CCG AAG TCC-3′ and the reverse primer (SEQ ID No. 4) 5′ GGA CTT CGG GGA TCC CCC CAT GGC-3′. PAGE purified primers were obtained from IDT. Single-stranded oligonucleotides were annealed and end-labeled using Gibco polynucleotide kinase and 50 microCurie gamma p³² ATP. Label was ethanol precipitated and resuspended in TE buffer.

Lysates were prepared from HeLA or 293 cells grown to 60-90% confluency in 6 well dishes. Cells were grown in DMEM growth medium or RPMI growth medium plus 20% fetal calf serum. They were washed twice in PBS and then stimulated with TNF alpha in RPMI for ten minutes at 37° C. After being washed once with PBS, lysates were made using 200 μL extraction buffer (as per Western Blot extraction buffer, described above). Lysates were cleared by centrifugation and stored at −80° C. before use.

For the gel-shift, 1 μL of extract was incubated with 2 μL of aptamer to which 1 uL of polyDIDC, 1 μL of probe, and 5 uL of binding buffer (20 mM TRIS pH 8, 200 mM KCl, 10 mM MgCl₂, 20% glycerol, 0.1% NP40, 1 mM DTT, 0.4 mg/mL bovine serum albumin) was added. Reactions occurred for 15 minutes at room temperature (RT). Samples were loaded onto 6% DNA Retardation Gels (Invitrogen) and run in 0.5×TBE at 150 V for 1.5 hours. Dried gels were placed on phosphoimager plates and radioactive bands were visualized by analysis with the Storm 860 (Molecular Dynamics, Sunnyvale, Calif.).

NF-κB Luciferase Assays. HEK293 cells were cultivated in DMEM supplemented in 10% fetal bovine serum in 96 well white plates at a density of 10,000 cells/well 24 hours before transfection. 5 ng of NF-κB TA Luciferase plasmid (Clontech, Palo Alto, Calif.) along with 80 ng of siRNA expression plasmid and 7SL expression plasmid were introduced into HEK 293 cells using fuGENE 6 (Roche). 24 hours later, cells were stimulated with human TNF-alpha at a concentration of 10 ng/ml for five hours at 37° C. Luminescence was measured using the Steady-Glo kit (Promega, San Luis Obispo, Calif.) and a TopCount Luminometer. In the combination experiments, 35 ng of the pAV7SL-derived plasmid, 55 ng of the p-Silencer-derived plasmid, and 5 ng of NF-κB TA Luciferase plasmid were used per well.

Knock-down of in vivo NF-κB activity with RNA Aptamers. Using in vitro selection methods, a 31-nt RNA aptamer that binds to the p50 subunit of NF-κB with high affinity has been identified (Cassiday et al. (2001) Biochemistry 40, 2433-2438). Furthermore, it has been shown that this aptamer can inhibit p50/p65 heterodimer DNA binding to its cognate DNA binding site in vitro and in a yeast three hybrid assay. To knock-down intracellular NF-κB activity, this aptamer was expressed in mammalian cells with various expression constructs. Since NF-κB exists both in the cytoplasm and the nucleus of mammalian cells, expression vectors designed to deliver the aptamer to either sub-cellular compartment were constructed. To deliver a-p50 to the cytoplasm, the vector pAV-7SL was used, thereby placing the aptamer within the cytoplasmic RNA 7SL and driving expression with the 7SL promoter (FIG. 2A). FIG. 2A shows a diagram of the transcript that would be generated from the aptamer expression constructs of the invention. FIG. 2A shows a schematic of a transcript generated from 7SL-NF-κB in which 7SL sequences are in black text, aptamer sequence shown underlined and terminator sequence shown in italics. FIG. 2B (Panel 1, left) shows a schematic of the HIV-1 TAR transcript (SEQ ID NO:7) consisting of a 59 nucleotide stable stem and loop structure. The DNA encoding the stem also encodes the bipartite IST element showing the IST sequence in normal text. The sequence most important for Tat binding is shown in italics. To make TAR-κB (FIG. 2B, Panel 2, right) (SEQ ID NO:5), the Tat binding site was replaced with the similarly shaped a-p50 aptamer which sequence is shown underlined.

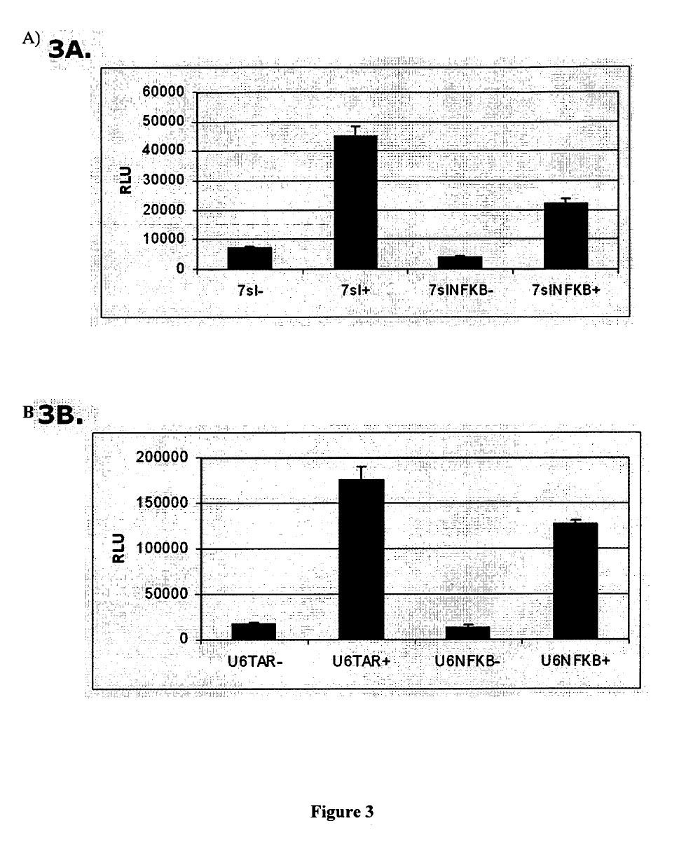

A new strategy was developed to deliver a-p50 to the nucleus at high levels. The vector U6-TAR expresses the HIV-1 TAR construct to high levels driven by the pol III U6 promoter. This vector also contains the HIV-1 IST element which has been shown to dramatically increase production of short transcripts from variety of promoters including the U6 promoter (Ratnasbapathy et al. (1990) Genes Dev. 4, 2061-2074; and Sheldon et al. (1993) Mol. Cell Biol. 13, 1251-1263). The dispensable top of the TAR which contains a natural aptamer that binds the HIV-1 Tat protein was replaced with a-p50 (FIG. 2B). Electrophoretic mobility shift assay (EMSA) studies using aptamers produced from these plasmids in vitro demonstrated that a-p50 inhibited p50 binding within the context of 7SL or TAR and the 7SL or TAR alone cannot. RT-PCR of extracts from cells transfected with these vectors with probes specific for the predicted RNA aptamers verify that both vectors expressed the aptamers=. In experiments performed, a-p50 retained NF-κB binding activity within the context of 7SL and TAR RNA vehicles. Lysates from TNF alpha stimulated 293 or HeLA cells were incubated with titrations of in vitro transcribed a-p50 or control aptamer in the presence of radiolabeled NF-κB DNA probe. Samples were run on 6% DNA retardation gels and then visualized after being exposed to phosphoimager plates. To determine whether expression of these aptamers can inhibit NF-κB function in mammalian cells, either the parent constructs (7SL or U6-TAR) or the respective a-p50 aptamer expression vectors were co-transfected into 293 cells with a NF-κB dependent luciferase reporter construct. Transfected cells were treated with TNF to enhance NF-κB activity and luciferase levels were measured 6 hours later. The results in FIGS. 3A and 3B show that in untreated cells co-transfected with 50 ngs of U6-TAR-a-p50 there is a small but reproducible down-regulation of NF-κB activity (11%) compared with cells transfect with the same amount of control plasmid. Stimulation of NF-κB activity by TNF-α treatment increases down-regulation to 29%. Similarly, in unstimulated cells co-transfected with 50 ngs of 7SL-κB NF-κB levels are reduced 39% compared to control plasmids and this reduction increases to 51% upon TNF stimulation. For both plasmids, transfecting more plasmid (up to 80 ngs) did not increase inhibition although transfecting smaller amounts showed dose responsive decreases in inhibition (data not shown).

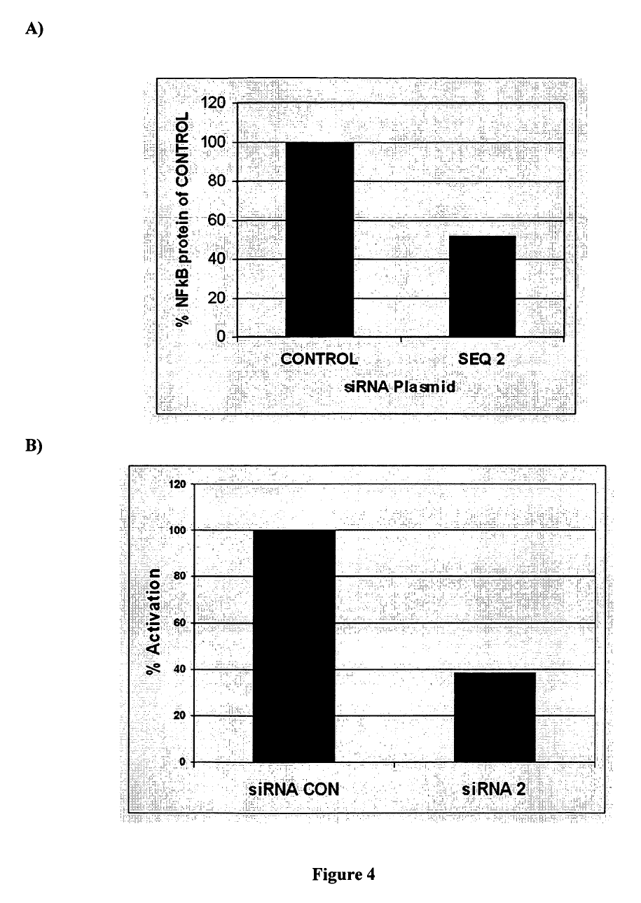

To determine whether siRNA could also inhibit NF-κB activity, a series of siRNAs were designed by the method of Tuschl as described above, transcribed in vitro, and transfected into 293 cells. 48 hours later the amount of NF-κB protein and a control were assayed by western blot and gel shift analysis. Of the sequences tested, siRNA2 yielded the best results, reducing protein levels by 61% (FIG. 4B). FIGS. 4A and 4B and shows that siRNA reduces NF-κB protein levels and activity as detected by various methods. A Western blot of extract from cells mock transfected (Zero A and B), with 50 nM in vitro transcribed single strand control RNA (ssRNA Control A and B) or 50 nM siRNA sequence number 2 (siRNA2 A and B) was performed (gel not shown). The blot was simultaneously treated with antibody to the p50 subunit of NF-κB and the TFIIB transcription factor (that should not be affected by siRNA treatment). FIG. 4A shows a bar graph of normalized results of Western blot of cells transfected with control p-Silencer-2.0 (Ambion) plasmid (CONTROL) or pSilencer-2.0-U6-siRNA2 (SEQ 2). Shown experiment is representative of three experiments. FIG. 4B shows a bar graph of results of NF-κB-dependent luciferase assay of cells transfected with NF-κB-dependent reporter plasmid and p-Silencer-2.0 (siRNA CON) or pSilencer-2.0-U6-siRNA2 (siRNA2, SEQ ID No. 3).

Next, siRNA's and aptamer's ability to reduce NF-κB activity in the NF-κB dependent luciferase assay was compared. To more easily compare the two methods, siRNA2 were delivered into the cell by expression from a plasmid. Expression of siRNA2 resulted in significant reductions in protein levels by western blot (FIG. 4B). Expression of siRNA2 also significantly reduced NF-κB activity (63%, FIG. 4B).

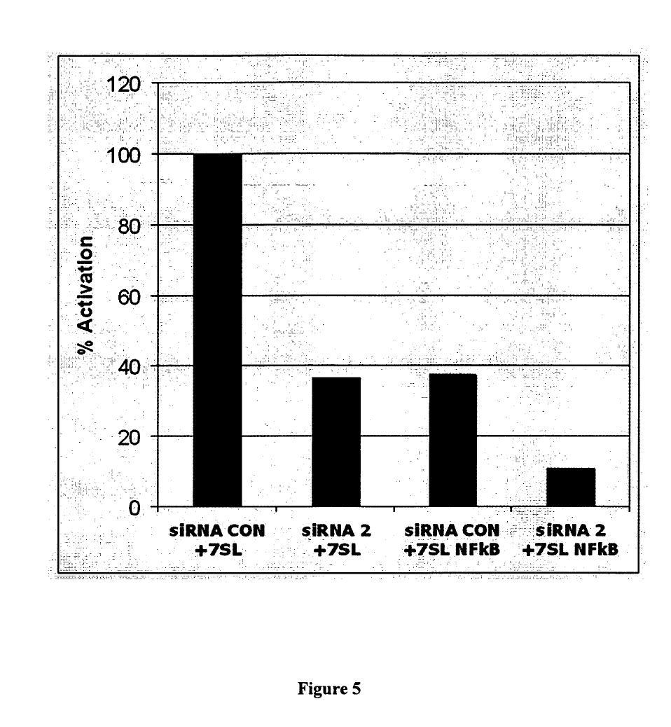

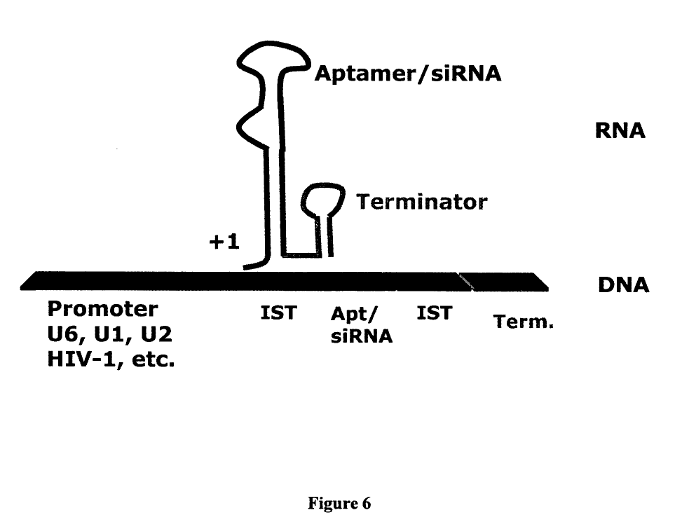

Aptamers and siRNA work at different levels of the gene expression pathway, thus combining the two methods provides stronger inhibition than either alone. This was shown by transfecting different combinations of the siRNA and aptamer expressing plasmids. As shown in FIG. 5, transfection of siRNA2 along with control 7SL plasmid resulted in 64% reduction in NF-κB activity compared to the control. Furthermore, transfection of 7SL-NF-κB along with siRNA control resulted in 62% repression. Transfection of both 7SL-NF-κB and siRNA2, however, led to 90% reduction of NF-κB activity. Thus, the most effective way to knock-down NF-κB activity using the two methods is to use them in combination. FIG. 5 shows that NF-κB activity is most significantly inhibited in the presence of both p50-specific siRNA and p50-specific aptamer. FIG. 5 shows the results of NF-κB dependent luciferase assay of cells transfected with NF-κB-dependent reporter plasmid along with control plasmids for both siRNA and aptamer expressors (siRNA CON+7SL), p-50 specific siRNA and aptamer control plasmid (siRNA2+7SL), p-50 specific aptamer+siRNA control plasmid (siRNA CON+7SL NF-κB), and plasmids expressing both p-50 specific siRNA and aptamer (siRNA 2+7SL NF-κB). FIGS. 6 and 7 are schematic representations of the intracellular expression cassettes of the invention. FIG. 6 shows an expression construct having a promoter region which includes, but is not limited, to U6, U1, U2, and HIV-1 promoter regions, an IST region, an aptamer or siRNA insert sequence, an IST region, and a transcription termination region. FIG. 7 shows another embodiment of the invention showing a possible tandem expression cassette for an aptamer and an siRNA sequences. This embodiment comprises a promoter region which includes, but is not limited to, U6, U1, U2, and HIV-1 promoter regions, an IST region, a first aptamer or siRNA sequence region, an IST region, a second aptamer or siRNA sequence region, an IST region, and a terminator region.

In summary, the methods of the present invention utilizing an aptamer that has been shown to inhibit the DNA binding activity of the p50 subunit of the NF-κB transcription factor in yeast (Lebruska, et al., (1999) Biochemistry, 38, 3168-3174) were used for the first time to knock-down NF-κB intracellular activity in mammalian cells. In addition, NF-κB activity was knocked-down with siRNA. Both aptamers and siRNAs were found to have knock-down activity in a similar manner, and interestingly, when used in combination the two methods work better than either method alone, leading to a 90% knock-down of activity.

The invention having now been described by way of written description and example, those of skill in the art will recognize that the invention can be practiced in a variety of embodiments and that the foregoing examples are for purposes of illustration and not limitation of the following claims.

1) An intracellular aptamer expression vector, comprising a transcription promoter region, an aptamer sequence region, an inducer of short transcripts (IST) sequence region, and a transcription termination region functionally linked to allow transcription.

2) The intracellular aptamer expression vector of claim 1, wherein the inducer of short transcript (IST) region is derived from HIV-1 trans-activation region (TAR) sequence.

3) The intracellular aptamer expression vector of claim 2, wherein the transcription promoter region is a pol III promoter region sequence selected from U1, and U6 transcription promoter regions.

4) The intracellular aptamer expression vector of claim 3, wherein the aptamer sequence region is an aptamer that binds to an intracellular protein.

5) The intracellular aptamer expression vector of claim 4, wherein the aptamer sequence region is an aptamer that binds to NF-κB.

6) The intracellular aptamer expression vector of claim 5, wherein the aptamer sequence is an aptamer specific to NF-κB p50.

7) The intracellular aptamer expression vector of claim 6, wherein the p50 aptamer specific to NF-κB has sequence SEQ ID No. 6.

8) The intracellular aptamer expression vector of claim 3, wherein the aptamer sequence region is up to 50 nucleotides long to prevent protein kinase response leading to cell death.

9) An intracellular small interfering RNA (siRNA) expression vector, comprising a transcription promoter region, an siRNA sequence region, an inducer of short transcripts (IST) sequence region, and a transcription termination region functionally linked to allow transcription.

10) The intracellular small interfering RNA (siRNA) expression vector of claim 8 wherein the small interfering RNA (siRNA) sequence region is the siRNA specific for Nuclear Factor κ B (NF-κB) of SEQ ID No. 2.

11) The intracellular small interfering RNA (siRNA) expression vector of claim 9, wherein the IST region is derived from HIV-1 trans-activation region (TAR) sequence.

12) The intracellular small interfering RNA (siRNA) expression vector of claim 10, wherein the transcription promoter region is a pol III promoter region sequence selected from U1, and U6 transcription promoter regions.

13) The intracellular small interfering RNA (siRNA) expression vector of claim 11

14) A small interfering RNA specific for Nuclear Factor κ B (NF-κB) of SEQ ID No. 2.

15) An intracellular nucleic acid expression vector for the tandem expression of siRNA and aptamers, comprising a transcription promoter region, an aptamer sequence region, an siRNA sequence region, at least one inducer of short transcripts (IST) sequence region, and a transcription termination region functionally linked to allow transcription.

16) The intracellular nucleic acid expression vector of claim 15, wherein said transcription promoter region is a pol III transcription promoter sequence region selected from U1 and U6 transcription promoter regions.

17) The intracellular nucleic acid expression vector of claim 15, wherein said aptamer is up to 50 nucleotides long to prevent protein kinase response leading to cell death.

18) The intracellular nucleic acid expression vector of claim 16, wherein said aptamer sequence region is an aptamer specific to NF-κB p50.

19) The intracellular nucleic acid expression vector of claim 17, wherein said siRNA is an siRNA specific for Nuclear Factor κ B (NF-κB) of SEQ ID No. 2.

20) The intracellular nucleic acid expression vector of claim 18 wherein said at least one inducer of short transcripts (IST) sequence region is derived from HIV-1 TAR sequence.

21) A method of maximizing gene knock-down in mammalian cells, comprising intracellular expression of an siRNA and an aptamer specific to a protein target expressed in said mammalian cell.

22) The method of claim 21 wherein said protein target is an intracellular protein target.

23) The method of claim 22 wherein said protein target is NF-κB protein.

24) The method of claim 21 wherein said protein target is an extracellular protein target.

25) The method of claim 21 wherein said siRNA and aptamer are specific for different protein targets involved in a disease pathogenesis.

26) A method of target validation by gene-knock down in mammalian cells comprising intracellular expression of an siRNA and an aptamer to knock down gene expression of a target protein expressed in said mammalian cell.

27) The method of claim 26 wherein said protein target is an intracellular protein target.

28) The method of claim 27 wherein said protein target is NF-κB protein.

29) The method of claim 26 wherein said protein target is an extracellular protein target.

30) The method of claim 26 wherein said siRNA and aptamer are to different protein targets involved in a disease pathogenesis.
